(19) United States
(12) Patent Application Publication
(10) Pub. No.: US 2012/0094915 A1
(43) Pub. Date: Apr. 19, 2012

(54) ANTI-MENTAL FATIGUE DRUG

(75) Inventors: Ran Emilie Yoshise, Hokkaido (JP); Hiroaki Matsuyama, Hokkaido (JP); Yukio Kadooka, Hokkaido (JP)

(73) Assignee: MEGMILK SNOW BRAND CO., LTD., Hokkaido (JP)

(21) Appl. No.: 13/264,548

(22) PCT Filed: Apr. 8, 2010
(86) PCT No.: PCT/JP2010/056350

§ 371 Date: Dec. 6, 2011

(30) Foreign Application Priority Data
Apr. 17, 2009 (JP) 2009-101017

(51) Int. Cl. A61K 38/40; A61P 25/00; C07K 14/79

(52) U.S. Cl. 514/17.7; 530/395

(57) ABSTRACT

To provide anti-mental fatigue drugs as well as foods, beverages, and fodders that can be taken daily and effective for relieving mental stress and inducing relaxation by intake. An anti-mental fatigue drug containing iron-binding lactoferrin as an active ingredient and foods, beverages, and fodders containing the drug.

### TECHNICAL FIELD

This invention relates to an anti-mental fatigue drug, which can relieve mental stress or have relaxing effects when administered orally. This invention is characterized by containing iron-binding lactoferrin (iron-bound lactoferrin) as an active ingredient.

### BACKGROUND ART

At present, we live in a social environment that causes us severe mental stress. Mental stress affects people from all walks of life, irrespective of age and sex, and even increases during menstruation or menopause in women. It occurs in situations where excellent results are expected, such as in athletics or in examinations, or where communication is required, such as in human interaction. Many methods of relieving mental stress, which is a great challenge facing modern society, have been proposed. Although various training methods (e.g., mental training) have been used to control mental stress, it takes enormous time and money to master them.

Drugs stimulating the central or sympathetic nervous system are known to influence or prolong one's maximum abilities. Examples include stimulants (e.g., amphetamines, caffeine, cocaine, and ephedrine), narcotic analgesics (e.g., morphine), and hormones and their analogs.

In addition, it is disclosed that chemically synthesized drugs such as tranquilizers, antianxiety drugs, or sleeping pills (hypnotics) are used to reduce mental fatigue.

Foods containing gamma-aminobutyric acid (GABA), lysine and arginine, theanine, and herbs have been reported to have relaxing effects on the mind and body and reducing mental stress (See for example PATENT LITERATURE 1, 2, and 3).

Lactoferrin has been disclosed as an immunostimulant in an anti-stress drug for aquatic animals (See for example PATENT LITERATURE 4). Additionally, an anti-stress drug (See for example PATENT LITERATURE 5) and an anti-fatigue drug (See for example PATENT LITERATURE 6) that contain lactoferrin as an active ingredient have been disclosed for terrestrial animals.

Anti-stress effects of lactoferrin have been examined in mature rats (See for example NON PATENT LITERATURE 1) in which the following mechanism of action is inferred: Intraperitoneally administered lactoferrin, transferred internally (directly or via cytokines), enhances the production of nitrogen monoxide in the brain and signal transduction of morphine-like opioid, and accordingly, suppresses secretion of stress hormones, which are released via the intermediate pituitary gland by stress transmitted to the cerebral cortex, resulting in reduced stress. Meanwhile, lactoferrin has been found to have no risk of dependence, unlike narcotics and exogenous opioids, because it does not bind to opioid receptors. Thus, the development of the combination of analgesics and side effect-free enteric-coated lactoferrin has also progressed, suggesting effects for reducing premenstrual irritability and menorrhalgia. In addition, its efficacy for phases 1 and 2 pains accompanying arthritis and osteosynthesis-related disease is disclosed (See for example PATENT LITERATURE 7).

### CITATION LIST

### Patent Literature

PATENT LITERATURE 1: JP 2003-252755 A
PATENT LITERATURE 2: WO2005/070408
PATENT LITERATURE 3: JP 2005-232045 A
PATENT LITERATURE 4: JP H11-514973 A
PATENT LITERATURE 5: JP 2001-354583 A
PATENT LITERATURE 6: JP 2007-22989 A
PATENT LITERATURE 7: JP 2008-44879 A

### Non Patent Literature

NON PATENT LITERATURE 1: Brain Res., Volume 68: Page 102, Year 2006

### SUMMARY OF INVENTION

### Technical Problem

Unfortunately, all stimulants (e.g., amphetamines, caffeine, cocaine, and ephedrine), narcotic analgesics (e.g., morphine), and hormones and their analogs have serious side effects and are prohibited from use as medication.

Moreover, chemically synthesized drugs such as tranquilizers, antianxiety drugs, or sleeping pills to relieve mental fatigue are problematic when taken daily on a long-term basis because of side effects and addiction. Furthermore, the necessity of a physician's prescription for these drugs prevents easy use or applying them to foods.

Foods containing GABA, lysine and arginine, theanine, and herbs reported in PATENT LITERATURE 1, 2, and 3 can only be used in limited foods and beverages because they have distinctive and strong odors or flavors. Moreover, problems such as complicated manufacturing methods and high cost have been raised.

PATENT LITERATURE 4 reported lactoferrin as an immunostimulant (more than 30 are proposed) contained in anti-stress drugs for aquatic animals. However, it is not indicated for terrestrial animals including human, but for freshwater and saltwater aquatic animals (especially, fish, shrimp, and invertebrates).

PATENT LITERATURE 5 and 6 demonstrated effects on stress not caused by mental load but by physical exercise. It is reported that stresses from physical exercise and mental load are mediated through different cerebral pathways, making a shared indication difficult. Other issues are limited versatility in terms of foods, beverages, or fodders and high cost because the effective dose of lactoferrin to exert physiological effects is extremely high.

PATENT LITERATURE 7 and NON PATENT LITERATURE 1 have presented the mental stress relief effect of lactoferrin, though it is restricted to mental load produced by pain. In addition, the experiment in mature rats selected administration via the direct intraperitoneal route or oral enteric-coated preparations to avoid decomposition of lactoferrin by gastric juice. For humans, the problems are that intraperitoneal administration is impossible and that enteric-coated preparation is difficult to implement in foods, beverages, and fodders. On the other hand, this invention is especially effective for stress from daily life and intellectual endeavors, in addition to having a different mechanism.

In conclusion, the problem to be resolved by this invention is to provide an anti-mental fatigue drug as well as foods, beverages, and fodders with similar functions, which terrestrial animals, including humans, can take daily and which is effective in relieving stress from mental load and induce relaxation.

### Solution to Problem

After deeply committing to solve the above problems, these inventors found that orally administered iron-binding lactoferrin demonstrates the effects of relaxation and causes reduction in mental fatigue in mental stress loading. Iron-binding lactoferrin is intended to improve heat and digestion tolerance by binding 3-200 iron molecules to 1 lactoferrin molecule. iron-binding lactoferrin is more stable than lactoferrin in the body, which eliminates the need to create enteric-coated preparations and enables a greater therapeutic effect with lesser amount. These inventors arrived at this invention upon discovering that it excels in solubility and preservation stability, with a wide range of properties, which allows application to foods, beverages, and fodders.

The preferred embodiments of this invention relating to the anti-mental fatigue drug are shown as follows:

(1) An anti-mental fatigue drug comprising iron-binding lactoferrin as the active ingredient.

(2) An anti-mental fatigue drug for oral ingestion comprising iron-binding lactoferrin as an active ingredient.

(3) Foods and beverages containing anti-mental fatigue drug according to item (1) or (2) above.

(4) Fodders containing anti-mental fatigue drug according to item (1) or (2) above.

Other preferred embodiments of this invention are shown as follows:

(a) A method of suppressing mental fatigue, comprising administering iron-binding lactoferrin.

(b) A method of suppressing mental fatigue, comprising orally administering iron-binding lactoferrin.

### Advantageous Effects of Invention

The invention of an anti-mental fatigue drug with iron-binding lactoferrin as the active ingredient and foods, beverages, and fodders containing iron-binding lactoferrin is effective in relieving mental stress.

### BRIEF DESCRIPTION OF DRAWINGS

FIG. 1 Change rate in α-wave area indicating relaxed state (Test Example 1).

FIG. 2 β-wave area indicating tensed state (Test Example 2).

FIG. 3 α-wave area indicating relaxed state (Test Example 2).

FIG. 4 Concentration of chromogranin A, a fatigue marker, in saliva (Test Example 2).

FIG. 5 Scores of “Tension-Anxiety,” “Depression-Dejection,” “Anger-Hostility,” “Vigor,” “Fatigue,” and “Confusion” quantified by POMS (Test Example 2).

### DESCRIPTION OF EMBODIMENTS

The embodiment of this invention will now be described in more detail as follows:

One molecule of lactoferrin is usually chelated with 2 atoms of iron, which means 1 gram of lactoferrin accommodates 1.4 mg of iron. On the other hand, one molecule of iron-binding lactoferrin in this invention can stably accommodate at least 3 and preferably 3 to 200 atoms of iron. By producing this kind of iron-binding lactoferrin, a large amount of iron can be accommodated by lactoferrin.

This type of iron-binding lactoferrin is known traditionally. For example, iron-lactoferrin obtained in which lactoferrin is dissolved in water, iron compounds are added, lactoferrin reacts with iron, and the iron in the solution attains a non-free state (JP H04-141067 A); lactoferrin powder obtained in which ferrate is added to the lactoferrin solution, alkali is added, and pH of the solution is increased to stably keep iron (JP H07-17875 A); heat-resistant iron-lactoferrin conjugate obtained in which iron is bound to the amino group of lactoferrin via bicarbonate ion (JP H06-239900 A); or iron-lactoferrin complex obtained in which a solution of carbonic acid and/or bicarbonic acid plus lactoferrin and a solution of iron are mixed (JP H07-304798 A). In addition, iron-lactoferrin degradation products are known.

Iron-binding lactoferrin may be derived from any of the above methods. iron-binding lactoferrin represents the binding substances of iron and lactoferrin, including iron-lactoferrin binding with or without a mediating substance, so that iron is not in an ionic state. Especially, the above “heat-resistant iron-lactoferrin conjugate” or “iron-lactoferrin complex” is preferable. Because iron-binding lactoferrin is characterized by absence of astringency or metallic taste of iron, it does not cause any problem in flavor.

When manufacturing iron-binding lactoferrin, we can use lactoferrin separated from secretions, for example, from mammalian milk, as raw material. Based on the fact that lactoferrin is a natural ingredient derived from milk and supported by our long-term experience with its use as food, its consumption is safe and will cause no side effects even with long-term intake. Thus, lactoferrin can be taken orally ad libitum.

Furthermore, transferrin from blood and organs and ovotransferrin from eggs can be used as well. Many methods to prepare large amounts of these lactoferrins are known, and the known methods are acceptable. Incompletely isolated lactoferrins, containing other ingredients are also acceptable. Lactoferrins produced by genetic manipulation from microorganisms, animal cells, and transgenic animals can also be used. Moreover, lactoferrin degraded by proteases, (e.g., trypsin, pepsin, and chymotrypsin) acids, or alkalis can also be used.

When iron-binding lactoferrin is manufactured, raw materials of iron include, for example, ferrous sulfate, ferrous gluconate, ferrous lactate, iron citrate, ferrous sodium citrate, ferrous ammonium citrate, ferrous pyrophosphate, ferric pyrophosphate, ferric chloride, ferric nitrate, and ferric sulfate.

Although the active ingredient of the anti-mental fatigue drug is iron-binding lactoferrin, other nutrients (e.g., calcium, magnesium, vitamin D, vitamin K, and oligosaccharides) can be added.

In addition, if necessary, other publicly known additives (e.g., diluents (excipients), disintegrators, binders, lubricants, antioxidants, coating agents, coloring agents (colorant), corrigents (correctives), surface active agents, and plasticizers) can be mixed to produce oral formulations (e.g., granules, powders, capsules, tablets, dry syrups, and liquids) in the usual manner. Excipients include mannitol, xylitol, methyl cellulose sodium, calcium hydrogen phosphate, wheat starch, rice starch, corn starch, potato starch, carboxymethyl starch sodium, dextrin, α-cyclodextrin, β-cyclodextrin, carboxyvinyl polymer, light anhydrous silicic acid, titanium oxide, magnesium aluminometasilicate, polyethylene glycol, and medium chain fatty acid triglyceride. Palatable drinks (solution form) can be prepared by adding other physiologically active ingredients, minerals, vitamins, nutrients, and flavorings as needed.

Iron-binding lactoferrin (iron-binding lactoferrin) can be used in liquid formulae immediately after preparation as well as added in powder form, obtained by freeze-drying or spray-drying.

The anti-mental fatigue effects are suppression of increased tension, depression, anger, fatigue, and confusion and decreased vigor; increasing the amplitude of α-waves, which indicates relaxed state brain waves; or decreasing the amplitude of β-waves, which indicates a state of tension, due to consumption of iron-binding lactoferrin. In addition, the effects include improvement in the above items when there is a lot of mental stress.

Mental stress is caused by factors such as, but not limited to, mental tension, repetitive work, intellectual labor, premenstrual anxiety and tension, and irritability, and includes any stressful feeling.

“Foods and beverages containing iron-binding lactoferrin as an active ingredient” produced by the above methods include any foods and beverages—iron-binding lactoferrin itself or foods and beverages containing iron-binding lactoferrin. Iron-binding lactoferrin can be added to any foods and beverages for eating or drinking or to raw materials of foods and beverages during manufacturing processes. Foods and beverages include dairy products (cheese, butter, and fermented milk), beverages (yogurt drink, coffee beverage, and fruit juice), confectioneries (jelly, pudding, cookie, biscuit, and wafer), and frozen foods.

This invention enables supplemental ingestion of iron as it contains iron. Although foods and beverages containing iron for supplementation have problems that iron accelerates oxidation and effects on flavor, this invention is helpful to concomitantly take iron, which is usually difficult to add to foods and beverages.

Next, “Anti-mental fatigue fodders containing iron-binding lactoferrin as an active ingredient” obtainable by the above methods are for livestock. It can be added to any fodder, as above with foods and beverages, and to raw materials during the manufacturing processes.

Dosage of iron-binding lactoferrin for anti-mental fatigue can be decided ad libitum, considering weight, sex, and age. The content for adults should be adjusted so that 10-6,000 mg/day of iron-binding lactoferrin can be taken. The amounts of 100-2,000 mg, 150-1,000 mg, and 200-900 mg are more preferable in ascending sequence. Effects are observed even with a dosage as low as this. The anti-mental fatigue substance iron-binding lactoferrin of this invention exerts its effects when it is orally ingested as an anti-mental fatigue drug or as foods, beverages, fodders containing the drug.

This invention, iron-binding lactoferrin as an anti-mental fatigue drug, is safe, and it is tasteless, odorless, and colorless as well as practically useful. In addition, because iron-binding lactoferrin comes in powder form, tolerates ingestion and heat well, and displays excellent solubility, it can be applied to many foods and beverages, so that it is available in daily activities. Thus, this invention contributes extraordinarily to our stressful society as an anti-mental fatigue drug as well as a food, beverage, or fodder containing the drug.

Examples and Test Examples are shown below to explain this invention in detail. These examples are for illustrative purposes only and are not intended to be limiting on the scope.

### Example 1

### Iron-Binding Lactoferrin Preparation 1

Ninty grams (90 g) of lactoferrin (TATUA), 20 g of ferric chloride hexahydrate, and 5 g of sodium bicarbonate were dissolved in 10 L of water to make a solution containing iron-binding lactoferrin. After the solution was desalinated and concentrated using an ultrafilter, which can remove substances with molecular weight of less than 5,000, water was added to make 10 L of iron-binding lactoferrin solution.

After this solution was freeze dried, it contained 70 atoms of iron per one molecule of lactoferrin, which was determined as iron-binding lactoferrin (70FeLF) powder. It could be used as an anti-mental fatigue drug in this invention.

### Test Example 1

### Relaxing Effects with Intake of Iron-Binding Lactoferrin

This study aimed to examine the relaxing effects of iron-binding lactoferrin after its intake. We measured brain waves (electroencephalogram) after intake of iron-binding lactoferrin obtained in EXAMPLE 1.

(Subjects)

We conducted studies in 45 healthy subjects of 18 years or older, who ingested control or iron-binding lactoferrin (70FeLF) diets obtained in EXAMPLE 1.

(Method)

We measured α-waves (brain waves indicating relaxed state) for 3 min by FM515A (Futek Electronics Co., Ltd.) in each subject at rest in a room (26° C.) not accessible externally. The results were analyzed by Parallax II (Futek Electronics Co., Ltd.). After administering control or iron-binding lactoferrin diets, we kept subjects at rest for about 15 min for digestion and absorption, and measured brain waves again for analysis. The findings of control and iron-binding lactoferrin diets are shown in TABLE 1.

TABLE 1

| Category | Raw material |
|---|---|
| Iron-binding | Iron-binding lactoferrin 823 mg, |
| lactoferrin diet | Water 50 mL |
| Control diet | Caramel powder 82 mg |
| | Water 50 mL |

Brain wave area (amplitude) is defined as measurements derived by cumulating α and β-waves per second for 3 min (measurement time), presented in μW.

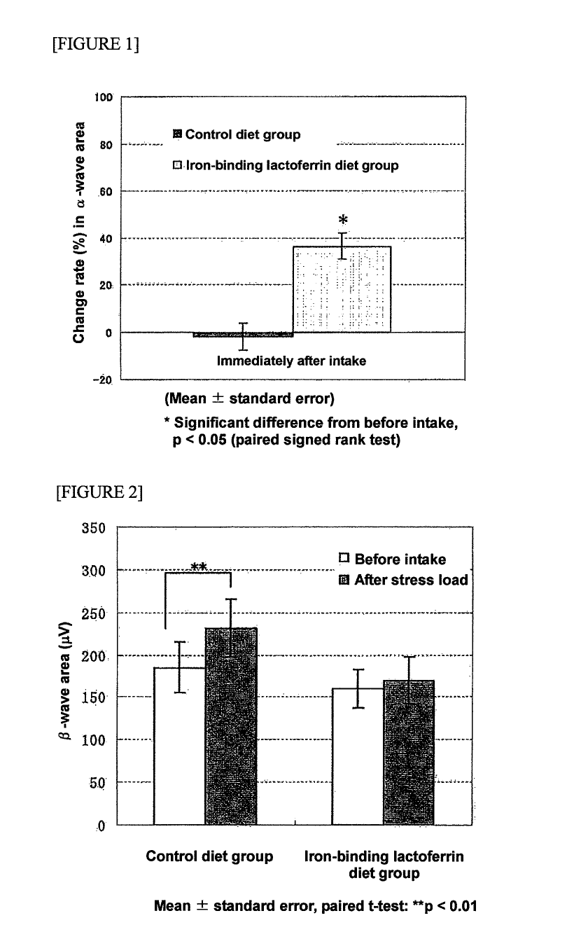

FIG. 1 shows the rate of change in brain wave areas between before and immediately after intake of control or iron-binding lactoferrin diets. While the control diet group showed no change in α-wave indicating a relaxed state, the iron-binding lactoferrin group revealed a significant increase from before intake. The results indicated that iron-binding lactoferrin has relaxing effects.

### Test Example 2

### Anti-Mental Fatigue Effects with Intake of Iron-Binding Lactoferrin

This study aimed to examine the anti-mental fatigue effects of iron-binding lactoferrin after its intake. After administering iron-binding lactoferrin obtained in EXAMPLE 1, we placed mental stress on subjects, measured brain waves and fatigue markers in saliva, and evaluated psychologic states, in order to evaluate the effects of reducing mental fatigue.

(Subjects)

Among the 45 healthy subjects of 18 years and older, we selected 24 subjects who were susceptible to fatigue caused by mental stress. “Susceptible to fatigue” is defined as those whose fatigue scores in Profile of Mood States (POMS; Kanekoshobo) increased by stress load. We conducted one test each, with control diet and with iron-binding lactoferrin (70FeLF) diet obtained in EXAMPLE 1 in all subjects.

(Method)

We measured two brain waves, α-waves (visible in relaxed states) and n-waves (visible in states of tension), by FM515A (Futek Electronics Co., Ltd.) in subjects at rest in a room (26° C.) not accessible externally. In addition, we collected saliva to measure chromogranin A and secretory IgA, which increases with acute stress. Chromogranin A is a fatigue marker, which increases only immediately after mental stress load, while secretory IgA is an index marker used to quantify decreased immunity caused by stress load. To evaluate transient mood, we performed a brief Japanese version of POMS (Profile of Mood States; Kanekoshobo) to generate scores in Tension, Depression, Anger, Fatigue, Confusion, and Vigor. POMS is an internationally acceptable scale that uses a questionnaire to objectively evaluate mood.

After administering control or iron-binding lactoferrin diets, we kept subjects at rest for 15 min for digestion and absorption. We then loaded mental stress using the Kraepelin test, which is a simple intellectual task, and immediately after, we measured brain waves, the fatigue markers in saliva, and POMS scores. We used Parallax II (Futek Electronics Co., Ltd.) for brain wave analysis. The compositions of control and iron-binding lactoferrin diets were same to those shown in TABLE 1.

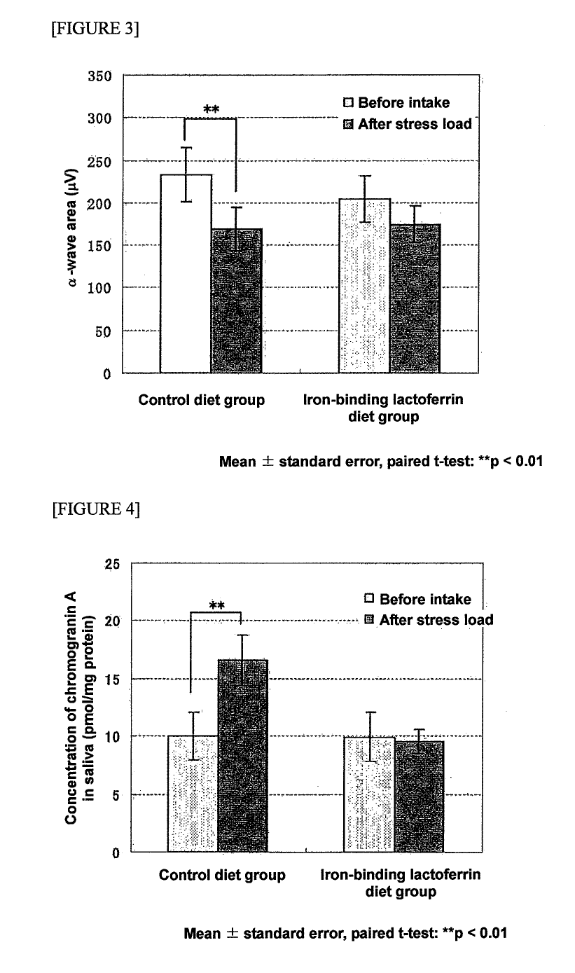

Brain wave areas before control or iron-binding lactoferrin diet and after stress load are shown in FIGS. 2 and 3. In the control diet group (control group), β-waves indicating states of tension significantly increased after stress loading (FIG. 2) and α-waves indicating relaxed states significantly decreased (FIG. 3). Meanwhile, the iron-binding lactoferrin group (lactoferrin group) showed no increased β-wave or decreased α-wave caused by stress load (FIGS. 2 and 3).

FIG. 4 shows chromogranin A concentration in saliva. An upward trend was observed in the control group before and after intake. Meanwhile, in the iron-binding lactoferrin group, no change was observed.

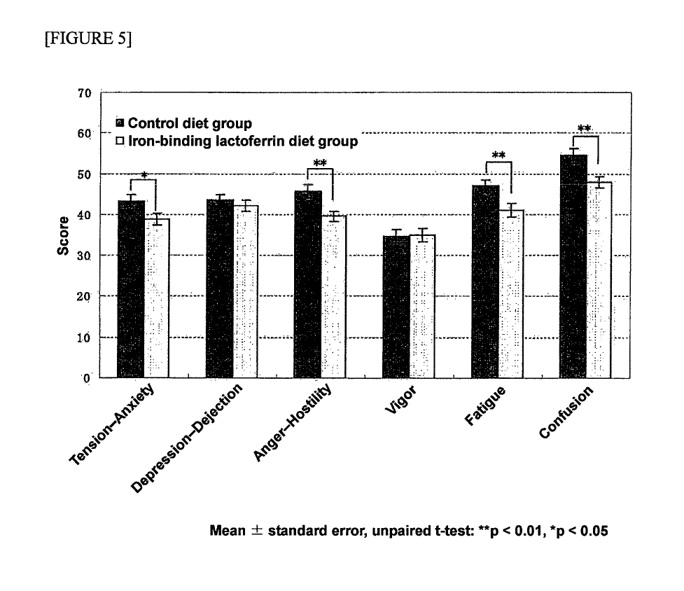

FIG. 5 indicates scores of Tension-Anxiety, Depression-Dejection, Anger-Hostility, Vigor, Fatigue, and Confusion after stress load, which were quantified by POMS for psychologic assessment. Scores for Tension-Anxiety, Anger-Hostility, Fatigue, and Confusion were significantly lower in the iron-binding lactoferrin group than in the control group.

These results indicate that iron-binding lactoferrin reduces fatigue caused by mental stress and improves mood and emotion.

### Example 2

### Iron-Binding Lactoferrin Preparation 2

Four hundred grams (400 g) of sodium bicarbonate was added to 2 L of water by stirring with a stirrer; Thus, a sodium bicarbonate supersaturated solution was prepared. To the solution another solution prepared by dissolving 90 g of commercially available lactoferrin (DMV) and 60 g of ferric chloride hexahydrate in 8 L of water was added while stirring, to make a solution containing iron-binding lactoferrin. After desalinating and concentrating the solution using an ultrafilter, which could remove substances with molecular weight of less than 5,000, water was added to make 10 L of iron-binding lactoferrin solution. After this solution was freeze dried, it contained 200 atoms of iron per one molecule of lactoferrin, which was determined as iron-binding lactoferrin (200FeLF) powder. It could be used as an anti-mental fatigue drug without modification.

Tests similar to Test Examples 1 and 2 with this anti-mental fatigue drug showed effects to decrease fatigue caused by mental stress.

### Example 3

We produced tablets by mixing iron-binding lactoferrin powder prepared in EXAMPLE 1 and calcium. Twenty percent (20%) of calcium carbonate, 10% of iron-binding lactoferrin powder, 40% of maltose, 16% of erythritol, 2% of sorbitol, 4% of flavoring, 0.5% of sweetener, 5% of excipient, and 2.5% of lubricant were mixed and tableted in the usual manner to produce tablets for anti-mental fatigue.

Tests similar to Test Examples 1 and 2 with this anti-mental fatigue tablet showed effects to decrease fatigue caused by mental stress.

### Example 4

We prepared soft drinks containing iron-binding lactoferrin prepared in EXAMPLE 2. To produce these anti-mental fatigue soft drinks, 0.1% of iron-binding lactoferrin powder, 0.12% of 50% lactic acid solution, 7.5% of maltitol, 0.2% of flavoring, and 92.08% of water were mixed and sterilized for 15 s at 90° C. using a plate sterilizer.

Tests similar to Test Examples 1 and 2 with this anti-mental fatigue soft drink showed effects to decrease fatigue caused by mental stress.

### Example 5

To produce an anti-mental fatigue milk-based beverage, iron-binding lactoferrin prepared in EXAMPLE 1 was added to raw milk to make an iron content of 3 mg/100 g, homogenized at 150 kgf/cm², and sterilized for 2 s at 130° C. using a plate sterilizer.

Tests similar to Test Examples 1 and 2 with this anti-mental fatigue milk-based beverage showed effects to decrease fatigue caused by mental stress.

### Example 6

We produced tablets containing iron-binding lactoferrin powder prepared in EXAMPLE 2 and calcium. To produce these tablets for anti-mental fatigue, 20% of calcium carbonate, 10% of iron-binding lactoferrin powder, 40% of maltose, 16% of erythritol, 2% of sorbitol, 4% of flavoring, 0.5% of sweetener, 5% of excipient, and 2.5% of lubricant were mixed, and tableted in the usual manner.

Tests similar to Test Examples 1 and 2 with this anti-mental fatigue tablet showed effects to decrease fatigue caused by mental stress.

### Example 7

### Manufacturing of Dog Food

To prepare an iron-binding lactoferrin powder, the size of the iron-binding lactoferrin powder prepared in EXAMPLE 1 was controlled with a 60-mesh sieve. Raw materials shown in TABLE 2 were mixed for producing anti-mental fatigue dog food.

Tests similar to Test Examples 1 and 2 with this anti-mental fatigue dog food showed effects to decrease fatigue caused by mental stress.

TABLE 2

| Iron-binding lactoferrin powder | 2.5 (weight %) |
|---|---|
| Powdered skimmed milk | 13.5 |
| Soybean dregs (soybean meal) | 12.0 |
| Soybean oil | 4.0 |
| Corn oil | 2.0 |
| Palm oil | 27.0 |
| Corn starch | 14.0 |
| Wheat flour | 9.0 |
| Wheat bran | 2.0 |
| Vitamins | 9.0 |
| Minerals | 2.0 |
| Cellulose | 3.0 |

### INDUSTRIAL APPLICABILITY

The invention of an anti-mental fatigue drug with iron-binding lactoferrin as the active ingredient and foods, beverages, and fodders containing iron-binding lactoferrin is effective in relieving mental stress.

1. An anti-mental fatigue drug comprising iron-binding lactoferrin as an active ingredient.

2. An anti-mental fatigue drug for oral ingestion comprising iron-binding lactoferrin as an active ingredient.

3. Foods and beverages containing the anti-mental fatigue drug according to claim 1.

4. Fodders containing the anti-mental fatigue drug according to claim 1.

5. Foods and beverages containing the anti-mental fatigue drug according to claim 2.

6. Fodders containing the anti-mental fatigue drug according to claim 2.

7. A method of suppressing mental fatigue, comprising administering iron-binding lactoferrin.

8. A method of suppressing mental fatigue, comprising orally administering iron-binding lactoferrin.
